(19) United States
(12) Patent Application Publication
(10) Pub. No.: US 2011/0178094 A1
(43) Pub. Date: Jul. 21, 2011

(54) Oral Formulation

(75) Inventors: Rene Holm, Jyllinge (DK); Christine Kau, Tastrup (DK); Birgitte Willumsen, Bronshoj (DK); Klaus Peter Hertel, Jystrup (DK); Christina Kurre Olsen, Smorum (DK); Lone Bruun, Solrod Strand (DK); Karina Krojer Soby, Jystrup (DK)

(73) Assignee: H. LUNDBECK A/S, Valby-Copenhagen (DK)

(21) Appl. No.: 13/119,846

(22) PCT Filed: Oct. 1, 2009
(86) PCT No.: PCT/DK2009/050258

§ 371 Date: Mar. 18, 2011

Related U.S. Application Data
(60) Provisional application No. 61/102,377, filed on Oct. 3, 2008.
(60) Provisional application No. 61/176,392, filed on May 7, 2009.

(30) Foreign Application Priority Data
Oct. 3, 2008 (DK) PA 200801392
May 7, 2009 (DK) PA200900591

(51) Int. Cl. A61K 31/496; C07D 241/04; A61P 25/18; A61P 25/24; A61P 25/22; A61P 25/06; A61P 25/16; A61P 25/36; A61P 25/34; A61P 25/32; A61P 25/28; A61P 25/00

(52) U.S. Cl. 514/252.12; 544/403

(57) ABSTRACT

The invention relates to a pharmaceutical composition intended for oral administration comprising low doses of 4-((1R,3S)-6-chloro-3-phenylindan-1-yl)-1,2,2-trimethylpiperazine and to a composition comprising the compound.

The present invention relates to a pharmaceutical composition intended for oral administration comprising low doses of 4-((1R,3S)-6-chloro-3-phenylindan-1-yl)-1,2,2-trimethylpiperazine. Moreover the invention relates to an improved binder in a composition comprising 4-((1R,3S)-6-chloro-3-phenylindan-1-yl)-1,2,2-trimethylpiperazine.

### BACKGROUND OF THE INVENTION

The compound which is the subject of the present invention (4-((1R,3S)-6-chloro-3-phenylindan-1-yl)-1,2,2-trimethylpiperazine) has the formula (I)

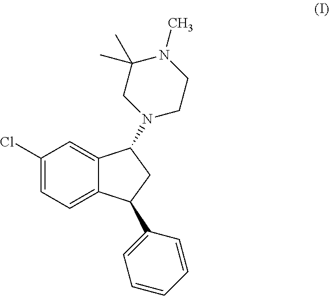

International patent publication No WO 2005/016900 discloses the compound of formula I (Compound I) as a free base and its corresponding succinate and malonate salts. The compound is reported to have high affinity for dopamine D1 and D2 receptors (antagonist), for the 5-HT₂ receptor (antagonist) and for α₁ adrenoceptors. In WO 2005/016900 the compound is suggested to be useful for treatment of several diseases in the central nervous system, including psychosis, in particular schizophrenia (positive, negative, and/or depressive symptoms) or other diseases involving psychotic symptoms, such as, e.g., Schizophrenia, Schizophreniform Disorder, Schizoaffective Disorder, Delusional Disorder, Brief Psychotic Disorder, Shared Psychotic Disorder as well other psychotic disorders or diseases associated with psychotic symptoms, e.g. mania in bipolar disorder. WO 2005/016900 also suggests the use of compound of formula I for treatment of anxiety disorders, affective disorders including depression, treatment of bipolar disorders, sleep disturbances, migraine, neuroleptic drug induced parkinsonism, as well as cocaine abuse, nicotine abuse, alcohol abuse and other abuse disorders. Other publications disclosing the compound of formula I and related compounds, having the above pharmacological profile, are EP 638 073; Bøgesø K. P.et al. J. Med. Chem., 1995, 38, page 4380-4392; and Bøgesø K. P. “Drug Hunting, the Medicinal Chemistry of 1-Piperazino-3-phenylindanes and Related Compounds”, 1998, ISBN 87-88085-10-4 (cf. e.g. compound 69 in table 3, p 47 and in table 9A, p 101).

### DESCRIPTION OF THE INVENTION

The compound of formula I is a putative antipsychotic compound with affinity for both dopamine D1 and D2 receptors. Preclinical experiments in rats using the condition avoidance response (CAR) model (Experimental procedure previously described in: Hertel P, Olsen C K, Arnt J. Repeated administration of the neurotensin analogue NT69L induces tolerance to its suppressant effect on conditioned avoidance behaviour. Eur J Pharmacol. 2002; 439(1-3):107-11.) have indicated that the compound of formula I possesses antipsychotic activity at very low levels of D2 receptor occupancy.

In a positron emission tomography (PET) study in healthy subjects using 11C-SCH23390 and ¹¹C-raclopride as D1 and D2 receptor tracers, it was found that the compound of formula I induces a D2 receptor occupancy of from 11 to 43% in the putamen when increasing the dose from 2 to 10 mg/day given daily for 18 days. Such level of D2 receptor occupancy is low in comparison with that of currently used antipsychotic drugs, which in general requires a D2 receptor occupancy around or exceeding 50% to be therapeutically effective (Stone J M, Davis J M, Leucht S, Pilowsky L S. Cortical Dopamine D2/D3 Receptors Are a Common Site of Action for Antipsychotic Drugs; An Original Patient Data Meta-analysis of the SPECT and PET In Vivo, Schizophr Bull. 2008 Feb. 26. [Epub in advance of print].). In the same PET study, it was found that the compound of formula I induces a D1 receptor occupancy increase from 32 to 69% in putamen when increasing the dose from 2 to 10 mg/day given daily for 18 days. Such high level of D1 occupancy is not generally seen with current used antipsychotic drugs (Farde L, Nordstrom A L, Wiesel F A, Pauli S, Halldin C, Sedvall G. Positron emission tomographic analysis of central D1 and D2 dopamine receptor occupancy in patients treated with classical neuroleptics and clozapine. Relation to extrapyramidal side effects. Arch Gen Psychiatry. 1992; 49(7):538-44.). Thus, the compound of formula I exhibits a unique ratio of D1 to D2 receptor occupancy at low daily doses.

Based on the above, it is expected that the compound of formula I have clinically significant therapeutic effects in patients with schizophrenia at doses (from 4 mg/day to 14 mg/day) that induce only a low level of D2 receptor occupancy. This might well be a consequence of the high D1 receptor occupancy and the unique ratio of D1 versus D2 receptor occupancy displayed by the compound of formula I. A low D2 receptor occupancy at therapeutically effective doses will be beneficial in terms of reduced tendency to induce troublesome side effects mediated by D2 receptor blockade, including extrapyramidal side effects and hyperprolactinemia.

The compound of formula I in a therapeutically effective amount of from 4-14 mg calculated as the free base is administered orally, and may be presented in any form suitable for such administration, e.g. in the form of tablets, capsules, powders, syrups or solutions. In one embodiment, a salt of the compound of formula I is administered in the form of a solid pharmaceutical entity, suitably as a tablet or a capsule.

Methods for the preparation of solid pharmaceutical compositions or preparations are well known in the art. Thus, tablets may be prepared by mixing the active ingredient with conventional adjuvants, fillers and diluents and subsequently compressing the mixture in a suitable tabletting machine. Examples of adjuvants, fillers and diluents comprise cornstarch, lactose, talcum, magnesium stearate, gelatine, gums, and the like. Typical fillers are selected from lactose, mannitol, sorbitol, cellulose and microcrystalline cellulose. Any other adjuvant or additive such as colourings, aroma, preservatives, etc, may also be used provided that they are compatible with the active ingredient.

As already indicated, the compound 4-((1R,3S)-6-chloro-3-phenylindan-1-yl)-1,2,2-trimethylpiperazine has the general formula (I)

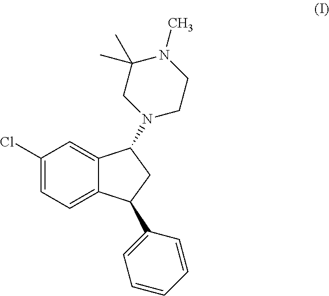

as used throughout the present description the term “compound of formula I” is intended to designate any form of the compound, such as the free base, pharmaceutically acceptable salts thereof, eg. pharmaceutically acceptable acid addition salts, such as succinate and malonate salts, hydrates or solvates of the free base or salts thereof, as well as anhydrous forms, amorphous forms, or crystalline forms.

The compound of formula I to be comprised in the composition of the present invention also comprises salts thereof, typically, pharmaceutically acceptable salts. Such salts include pharmaceutical acceptable acid addition salts. Acid addition salts include salts of inorganic acids as well as organic acids. Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like.

Further, the compound of formula I may exist in unsolvated form, as well as in solvated forms with pharmaceutically acceptable solvents such as water, ethanol and the like. In general, solvated forms are considered to be equivalent to unsolvated forms for the purposes of this invention.

The present invention relates to a pharmaceutical composition comprising the compound of formula (I)

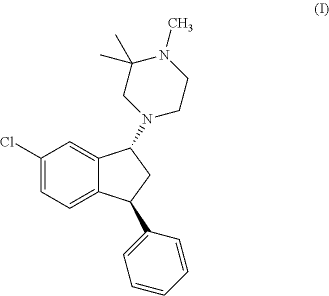

in a therapeutically effective amount of from 4-14 mg calculated as the free base.

In a further embodiment, the composition comprising the compound of formula I is for treatment of cognitive dysfunction, schizophrenia, Schizophreniform Disorder, Schizoaffective Disorder, Delusional Disorder, Brief Psychotic Disorder, Shared Psychotic Disorder, mania in bipolar disorder, anxiety disorders, depression, maintenance of bipolar disorders, sleep disturbances, migraine, neuroleptic-induced parkinsonism, or cocaine abuse, nicotine abuse, or alcohol abuse. Typical use of the composition of the invention is in the treatment of schizophrenia, such as positive symptoms of schizophrenia, or cognitive dysfunction in schizophrenia.

In a further aspect the present invention relates to use of a compound of formula (I) for the preparation of a medicament for treatment of cognitive dysfunction, schizophrenia, Schizophreniform Disorder, Schizoaffective Disorder, Delusional Disorder, Brief Psychotic Disorder, Shared Psychotic Disorder, mania in bipolar disorder, anxiety disorders, depression, maintenance of bipolar disorders, sleep disturbances, migraine, neuroleptic-induced parkinsonism, or cocaine abuse, nicotine abuse, or alcohol abuse, wherein the compound of formula I is present in a therapeutically effective amount of from 4-14 mg calculated as the free base.

In a further aspect the present invention also relates to a method of treating cognitive dysfunction, schizophrenia, Schizophreniform Disorder, Schizoaffective Disorder, Delusional Disorder, Brief Psychotic Disorder, Shared Psychotic Disorder, mania in bipolar disorder, anxiety disorders, depression, maintenance of bipolar disorders, sleep disturbances, migraine, neuroleptic-induced parkinsonism, or cocaine abuse, nicotine abuse, or alcohol abuse, comprising administering a therapeutically effective amount of from 4-14 mg calculated as the free base of the compound of formula Ito a patient in need thereof.

In an embodiment of the composition, the use, or the method of treatment of the invention, the compound of formula I is formulated for oral administration, such as a tablet or capsule, typically a tablet. The composition, such as a tablet, is typically for oral administration once daily.

In a further embodiment of the composition, the use, or the method of treatment, the compound of formula I is in the form of a succinate or malonate salt. Typically, the succinate salt.

In further embodiments of the composition, use, or method of treatment, the amount of the compound of formula (I) is from 4-12 mg.

In further embodiments of the composition, use, or method of treatment, the amount of the compound of formula (I) is from 5-14 mg.

In further embodiments of the composition, use, or method of treatment, the amount of the compound of formula (I) is from 4-6 mg, such as 5 mg.

In further embodiments of the composition, use, or method of treatment, the amount of the compound of formula (I) is from 6-8 mg, such as 7 mg.

In further embodiments of the composition, use, or method of treatment, the amount of the compound of formula (I) is from 8-10 mg.

In further embodiments of the composition, use, or method of treatment, the amount of the compound of formula (I) is from 10-12 mg.

In further embodiments of the composition, use, or method of treatment, the amount of the compound of formula (I) is from 12-14 mg, such as 14 mg.

In further embodiments of the composition, use, or method of treatment, the amount of the compound of formula (I) is from 5-7 mg.

In further embodiments of the composition, use, or method of treatment, the amount of the compound of formula (I) is from 7-9 mg.

In further embodiments of the composition, use, or method of treatment, the amount of the compound of formula (I) is from 9-11 mg, such as 10 mg

In further embodiments of the composition, use, or method of treatment, the amount of the compound of formula (I) is from 11-13 mg.

When the invention relates to the use or the method of treatment then the dose indicated above of from 4-14 mg, such as 5 mg, 7 mg, 10 mg, or 14 mg, is on a daily basis.

In a further embodiment of the composition, the use, or the method of treatment, the composition further comprises povidone, such as Kollidone 30 (CAS-No. 94800-10-9), or copovidone, such as Kollidone VA64 (CAS-No. 25086-89-9), as a binder. The binder is typically present in a concentration range of from 2-10% (w/w), such as 2-4%, 4-6%, 6-8%, 8-10%, 2-8%, 4-8%, 4-10%, or 6-10% (w/w).

In a further aspect the present invention also relates to a pharmaceutical composition comprising the compound of formula (I)

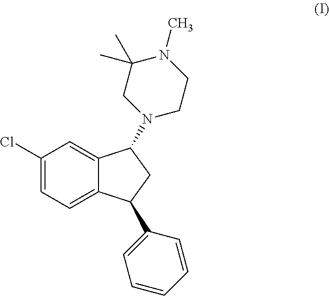

and povidone or copovidone as binder. Typically the binder is Kollidone VA64.

In an embodiment the binder is present in a concentration range of from 2-10% (w/w). Typically in a concentration range of from 2-4%, 4-6%, 6-8%, or 8-10% (w/w). When the binder is povidone or copovidone typical fillers are selected from calcium hydrogen phosphate lactose, mannitol, sorbitol, cellulose and microcrystalline cellulose, and preferably lactose, mannitol, sorbitol, cellulose and microcrystalline cellulose, such as lactose. In an embodiment the filler, such as anyone of the above, is in a concentration range of from 15-50% (w/w). Typically, the filler, such as anyone of lactose, mannitol, sorbitol, cellulose and microcrystalline cellulose, is in a concentration range of from 15-25%, 20-50%, 30-45% (w/w).

In a further embodiment of the composition the compound of formula (I) is in the form of the succinate salt.

Experimental

The safety and efficacy of the compound of formula I in schizophrenic patient will be investigated by standard measures of efficacy (including the Positive and Negative Syndrome Scale [PANSS] and the Clinical Global Impressions scale [CGI]) and safety. After a screening period, eligible patients will be randomised in a 2:1 ratio to blinded treatment with either the compound of formula I (e.g. at doses of 5, 7, 10 and 14 mg/day) or placebo for 8 weeks. The study includes 5 parts with increasing doses of the compound of formula I and a decision to initiate the next dose level will be based on safety and tolerability assessment based on the previous part of the study. The efficacy and the safety of the compound of formula I will be evaluated in comparison to the pooled placebo group from all parts of the study.

Efficacy on Cognitive Deficits in Schizophrenia

The compound of formula I has been shown to possess cognition enhancing properties in preclinical models of cognitive dysfunctions. It is believed that the 5-HT6 receptor affinity of the compound of formula I is involved in the precognitive effects of the compound. Furthermore, it is believed that such pro-cognitive effect of the compound of formula I will be evident at a low level of D2 receptor occupancy, which is beneficial in terms of the side-effect profile.

The effect of the compound of formula I on cognitive deficits in schizophrenic patients will be assessed in a clinical trial where eligible patients will be randomised in a 1:1 ratio to blinded treatment with flexible doses of either the compound of formula I (5 to 7 mg/day) or olanzapine (10 to 15 mg/day) for 12 weeks. The efficacy of the compound of formula I on cognitive symptoms will be assessed using the Brief Assessment of Cognition in Schizophrenia (BACS) scale (Keefe R S, Goldberg T E, Harvey P D, Gold J M, Poe M P, Coughenour L. The Brief Assessment of Cognition in Schizophrenia: reliability, sensitivity, and comparison with a standard neurocognitive battery. Schizophr Res. 2004; 68(2-3):283-97.i. Schizophr Res. 2004; 68(2-3):283-97.).

### EXAMPLE 1

### Preparation of Immediate Release Film-Coated Tablet Intended for Oral Administration I

Pharmaceutical Development

A study of the compatibility of the excipients and compound of formula I demonstrated that the components used in the tablet formulation were compatible with the compound. Based on this, a traditional wet granulation, tabletting and film-coating process was developed using standard methods and excipients.

Description of Drug Product

The compound of formula I is formulated as immediate release film-coated tablet intended for oral administration. Tablets containing compound of formula I in this example are made in two strengths, 5 and 7 mg. The product containing compound of formula I is a white film-coated tablet encapsulated in a brownish red hard capsule. Other strengths, such as 4, 6, 8, 9, 10, 11, 12, 13, or 14 mg, may be prepared in the same manner.

Composition

The compositions of the tablets 5 mg and 7 mg are given below in Table 1.

TABLE 1
Composition of tablets 5 mg and 7 mg

| | Quantity per Unit | | | | | Reference to |
|---|---|---|---|---|---|---|
| Name of Ingredient | 5 mg | | 7 mg | | Function | Standard¹ |
| DRUG SUBSTANCE | | | | | | |
| compound of formula I succinate | 6.665 | mg | 9.331 | mg | Active ingredient | In-house spec. |
| corresponding to compound of | 5 | mg | 7 | mg | | |
| formula I | | | | | | |
| EXCIPIENTS | | | | | | |
| Tablet core: | | | | | | |
| Calcium hydrogen phosphate, | 37.990 | mg | 36.213 | mg | Filler | Ph. Eur. |
| anhydrous | | | | | | |
| Maize starch | 18.995 | mg | 18.106 | mg | Filler | Ph. Eur. |
| Copovidone | 3.35 | mg | 3.35 | mg | Binder | Ph. Eur. |
| Water, purified² | q.s. | | q.s. | | Granulation liquid | Ph. Eur. |
| Cellulose, microcrystalline | 25 | mg | 25 | mg | Filler | Ph. Eur. |
| Croscarmellose sodium | 3 | mg | 3 | mg | Disintegrant | Ph. Eur. |
| Talc | 4 | mg | 4 | mg | Lubricant | Ph. Eur. |
| Magnesium stearate | 1 | mg | 1 | mg | Lubricant | Ph. Eur. |
| Weight of each tablet core | 100 | mg | 100 | mg | | |
| Film-coating: | | | | | | |
| Opadry Y-1-7000 white | | | | | | |
| consisting of: | | | | | | |
| Hypromellose (5 mPa · s.) | 1.563 | mg | 1.563 | mg | Film former | Ph. Eur. |
| Macrogol 400 | 0.156 | mg | 0.156 | mg | Plasticizer | Ph. Eur. |
| Titanium dioxide (E171) | 0.781 | mg | 0.781 | mg | Pigment | Ph. Eur. |

| | | | | |
|---|---|---|---|---|
| Water, purified² | q.s. | q.s. | Solvent | Ph. Eur. |

| | | | | |
|---|---|---|---|---|
| Weight of each film-coated tablet | 102.5 | mg | 102.5 | mg |

| | | | | |
|---|---|---|---|---|
| Magnesium stearate | q.s. | q.s. | Lubricant | Ph. Eur. |

¹The current pharmacopoeia is used
²Volatile material

The batch compositions for a representative batch size of 10,000 tablets are presented in Table 2.

TABLE 2
Batch composition for film-coated tablets (Batch size 10,000 tablets)

| | Strength | | | |
|---|---|---|---|---|
| | 5 mg | | 7 mg | |
| | | % w/w | | % |
| | | (per | | w/w |
| | Quantity | tablet | Quantity | (per |
| Ingredients | (g) | core) | (g) | tablet core) |
| Tablet core: | | | | |
| compound of formula I | 66.65 | 6.665 | 93.31 | 9.331 |
| succinate | | | | |
| Calcium hydrogen | 379.90 | 37.990 | 362.13 | 36.213 |
| phosphate, anhydrous | | | | |
| Maize starch | 189.95 | 18.995 | 181.06 | 18.106 |
| Copovidone | 33.5 | 3.35 | 33.5 | 3.35 |
| Water, purified¹ | q.s. | — | q.s. | — |
| Cellulose, | 250 | 25 | 250 | 25 |
| microcrystalline | | | | |
| Croscarmellose sodium | 30 | 3 | 30 | 3 |
| Talc | 40 | 4 | 40 | 4 |
| Magnesium stearate | 10 | 1 | 10 | 1 |
| Weight of tablet core | 100 mg | | 100 mg | |
| Film coating: | | | | |
| Opadry Y-1-7000 white | 25 | 2.5 | 25 | 2.5 |
| Water, purified¹ | q.s. | — | q.s. | — |

| | | |
|---|---|---|
| Weight of film-coated | 102.5 mg | 102.5 mg |
| tablet | | |

Description of Manufacturing Process and Process Controls

The method of granulation is a traditional wet granulation process using copovidone (Kollidone VA64) as a dry binder and water as granulation liquid. In the 10-litre PMA1 high shear mixer the process is as follows for a 2 kg batch:

Mix compound of formula I succinate, anhydrous calcium hydrogen phosphate, maize starch and copovidone for 2 minutes at 500 rpm.

Add purified water to initiate agglomeration.

Granulate at 800 rpm for approximately 4 minutes, so a suitable granule size is achieved.

Sieve the wet granules.

Dry the granules in a tray dryer at 50° C., until the product has a relative humidity (RH) of 25-55%RH.

Sieve the dried granules.

Mix the granules with microcrystalline cellulose, croscarmellose sodium and talc in a mixer.

Ad magnesium stearate to the mixer and mix.

Compress the granulate into tablets on a tablet compressing machine.

Film-coat the tablet cores in a film coater, using the process parameters given in table 3.

TABLE 3
Equipment and process conditions for the coating process.

| | | Spray | Inlet air | | |
|---|---|---|---|---|---|
| | Load | rate | flow | Inlet air | Outlet air |
| Equipment | (g) | (g/min) | (m³/h) | temp. (° C.) | temp. (° C.) |
| Compu Lab | 1360-1500 | 10 | 500 | 60 | 58 |
| 15″ | | | | | |

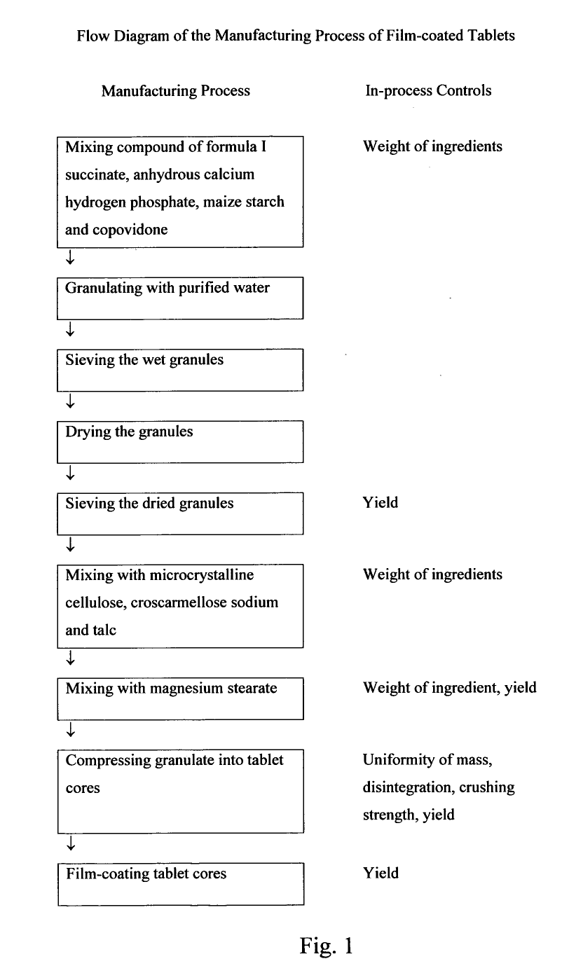

A flow diagram of the manufacturing process and process controls is shown in FIG. 1.

Unexpected Effects of Binder in the Tablet Formulation

In order to optimise the agglomeration process, two different tablet formulations was produced and their effect on the chemical stability of compound of formula I was evaluated. The composition of these tablets are given in table 4, and the manufacturing process, was similar to the one described above:

TABLE 4
Batch composition of film-coated tablets with 2 different binders (Batch
size 10,000 tablets)

Strength
2.5 mg

| | % w/w (per | % w/w (per |
|---|---|---|
| Ingredients | tablet core) | tablet core) |
| Tablet core: | | |
| compound of formula I | 2.67 | 2.67 |
| succinate | | |
| Calcium hydrogen | 40.66 | 40.66 |
| phosphate, anhydrous | | |
| Maize starch | 20.33 | 20.33 |
| Copovidone | 3.3 | 0.0 |
| Maltodextrin | 0.00 | 3.35 |
| Water, purified¹ | — | — |
| Cellulose, microcrystalline | 26.0 | 26.0 |
| Croscarmellose sodium | 3.0 | 3.0 |
| Talc | 3.0 | 3.0 |
| Magnesium stearate | 1.0 | 1.0 |

| | |
|---|---|
| Weight of tablet core | 125 mg |

The use of copovidone as binder leads to tablets with better pharmaceutical technical properties, e.g. the cabability of producing harder tablets with low loss on friability without compromising the disintegration time, as demonstrated in table 5:

TABLE 5
Comparision of pharmaceutical technical data for tablets containing
compound of formula I succinate with the composition given in table 4

| Copovidone | | | Maltodextrin | | |
|---|---|---|---|---|---|
| Applied | | | Applied | | |
| compression | Friability | Disintegration | compression | | |
| force (N) | (%, w/w) | time | force (N) | Friability (%) | |
| 86 | 0.14 | 44 sec | 36 | 0.69 | 43 sec |
| 108 | 0.16 | 1 min 14 sec | 47 | 0.51 | 1 min 13 sec |
| 120 | 0.18 | 1 min 52 sec | 51 | 0.43 | 1 min 42 sec |
| 130 | 0.22 | 2 min 09 sec | 59 | 0.23 | 1 min 59 sec |

Furthermore, the difference in binder lead to surprising stability differences as demonstrated in table 6

TABLE 6
Decomposition of compound of formula I succinate, in formulations
where maltodextrin and copovidon are used as binder, composition
of tablets given in table 4.

Total decomposition (%) of
compound of formula I

| Treatment | Copovidone | Maltodextrin |
|---|---|---|
| Initial analysis | <0.05 | <0.05 |
| After autoclavation | 0.91 | 1.1 |
| 80° C. for 48 hours | 0.99 | 2.0 |
| 80° C. for 120 hours | 1.4 | 3.7 |
| 40° C./75% RH for 3 | <0.05 | <0.05 |
| weeks | | |
| 60° C. for 3 weeks | 0.95 | 1.41 |

### EXAMPLE 2

### Preparation of Immediate Release Film-Coated Tablet Intended for Oral Administration II

Pharmaceutical Development

A study of the compatibility of the excipients and Compound I demonstrated that the components used in the tablet formulation were compatible with the compound. Based on this, a traditional wet granulation, tabletting and film-coating process was developed using standard methods and excipients.

Description of Drug Product

Compound I is formulated as immediate release film-coated tablet intended for oral administration. Tablets containing compound of formula I in this example are made in two strengths, 2.5 and 5 mg. The product containing compound of formula I is a white film-coated tablet encapsulated in a brownish red hard capsule. Other strengths, such as 2, 3, 4, 6, 7, 8, 9, 10, 11, 12, 13, or 14 mg, may be prepared in the same manner.

Composition

The compositions of the tablets 2.5 mg and 5 mg are given below in Table 7.

TABLE 7
Composition of tablets 2.5 mg and 5 mg (calcium phosphate form.)

| | Quantity per Unit | | | | | Reference to |
|---|---|---|---|---|---|---|
| Name of Ingredient | 2.5 mg | | 5 mg | | Function | Standard¹ |
| DRUG SUBSTANCE | | | | | | |
| Compound I, succinate | 3.333 | mg | 6.667 | mg | Active ingredient | In-house spec. |
| Corresponding to Compound I | 2.5 | mg | 5 | mg | | |
| EXCIPIENTS | | | | | | |
| Tablet core: | | | | | | |
| Calcium hydrogen phosphate, | 40.000 | mg | 80.000 | mg | Filler | Ph. Eur. |
| anhydrous | | | | | | |
| Maize starch | 20.000 | mg | 40.000 | mg | Filler | Ph. Eur. |
| Copovidone | 5.00 | mg | 10.00 | mg | Binder | Ph. Eur. |
| Water, purified² | q.s. | | q.s. | | Granulation liquid | Ph. Eur. |
| Cellulose, microcrystalline | 26.17 | mg | 52.34 | mg | Filler | Ph. Eur. |
| Croscarmellose sodium | 3 | mg | 6 | mg | Disintegrant | Ph. Eur. |
| Talc | 1.5 | mg | 3 | mg | Lubricant | Ph. Eur. |
| Magnesium stearate | 1 | mg | 2 | mg | Lubricant | Ph. Eur. |
| Weight of each tablet core | 100 | mg | 200 | mg | | |
| Film-coating: | | | | | | |
| Opadry Y-1-7000 white | | | | | | |
| consisting of: | | | | | | |
| Hypromellose (5 mPa · s.) | 1.563 | mg | 3.126 | mg | Film former | Ph. Eur. |
| Macrogol 400 | 0.156 | mg | 0.312 | mg | Plasticizer | Ph. Eur. |
| Titanium dioxide (E171) | 0.781 | mg | 1.562 | mg | Pigment | Ph. Eur. |

| | | | | |
|---|---|---|---|---|
| Water, purified² | q.s. | q.s. | Solvent | Ph. Eur. |

| | | | | |
|---|---|---|---|---|
| Weight of each film-coated tablet | 102.5 | mg | 205 | mg |

| | | | | |
|---|---|---|---|---|
| Magnesium stearate | q.s. | q.s. | Lubricant | Ph. Eur. |

¹The current pharmacopoeia is used
²Volatile material

The batch compositions for a representative batch size of 10,000 tablets are presented in Table 8.

TABLE 8
Batch composition for film-coated tablets (Batch size 10,000 tablets)

| | Strength | | | |
|---|---|---|---|---|
| | 2.5 mg | | 5 mg | |
| | | % w/w | | % w/w |
| | Quantity | (per tablet | Quantity | (per tablet |
| Ingredients | (g) | core) | (g) | core) |
| Tablet core: | | | | |
| Compound of formula I | 33.33 | 3.333 | 66.67 | 3.333 |
| succinate | | | | |
| Calcium hydrogen | 400.00 | 40.000 | 800.00 | 40.000 |
| phosphate, anhydrous | | | | |
| Maize starch | 200.00 | 20.000 | 400.00 | 20.000 |
| Copovidone | 50.0 | 5.00 | 100.0 | 5.00 |
| Water, purified¹ | q.s. | — | q.s. | — |
| Cellulose, | 261.7 | 26.17 | 523.4 | 26.17 |
| microcrystalline | | | | |
| Croscarmellose sodium | 30 | 3 | 60 | 3 |
| Talc | 15 | 1.5 | 30 | 1.5 |
| Magnesium stearate | 10 | 1 | 20 | 1 |
| Weight of tablet core | 100 mg | | 200 mg | |
| Film coating: | | | | |
| Opadry Y-1-7000 white | 25 | 2.5 | 50 | 2.5 |
| Water, purified¹ | q.s. | — | q.s. | — |

| | | |
|---|---|---|
| Weight of film-coated | 102.5 mg | 205 mg |
| tablet | | |

Manufacturing process and process controls is as in Example 1.

A flow diagram of the manufacturing process and process controls is shown in FIG. 1.

Unexpected Effects of Binder in the Tablet Formulation II

In order to optimise the agglomeration process, one tablet formulation (2.5 mg) for each binder was produced and the effect of binder on the chemical stability of Compound I was evaluated. The composition of these tablets is given in table 9, and the manufacturing process, was similar to the one described above.

TABLE 9
Batch composition of film-coated tablets with 7 different binders (Batch
size 10,000 tablets)
Strength 2.5 mg

| | % w/w (per | % w/w (per | % w/w (per | % w/w (per |
|---|---|---|---|---|
| | tablet core) | tablet core) | tablet core) | tablet core) |
| | Formulation no.: | | | |
| Ingredients | 1 | 2 | 3 | 4 |
| Tablet core: | | | | |
| Compound of formula I | 3.33 | 3.33 | 3.33 | 3.33 |
| succinate | | | | |
| Calcium hydrogen | 40.66 | 40.66 | 40.66 | 40.66 |
| phosphate, anhydrous | | | | |
| Maize starch | 20.33 | 20.33 | 20.33 | 20.33 |
| Pregelatinized starch | 5.0 | 0.0 | 0.0 | 0.0 |
| Hypromellose | 0.0 | 5.0 | 0.0 | 0.0 |
| Povidone | 0.0 | 0.0 | 5.0 | 0.0 |
| Methylcellulose | 0.0 | 0.0 | 0.0 | 5.0 |
| Water, purified¹ | — | — | — | — |
| Cellulose, microcrystalline | 25.2 | 25.2 | 25.2 | 25.2 |
| Croscarmellose sodium | 3.0 | 3.0 | 3.0 | 3.0 |
| Talc | 1.5 | 1.5 | 1.5 | 1.5 |
| Magnesium stearate | 1.0 | 1.0 | 1.0 | 1.0 |

| | |
|---|---|
| Weight of tablet core | 100 mg |

| | % w/w (per tablet | | % w/w (per tablet | | % w/w (per tablet | |
|---|---|---|---|---|---|---|
| | core) | | core) | | core) | |
| | Formulation no.: | | | | | |
| Ingredients | 5 | | 6 | | 7 | |
| Tablet core: | | | | | | |
| compound of | 3.33 | | 3.33 | | 2.67 | |
| formula I succinate | | | | | | |
| Calcium hydrogen | 40.66 | | 40.00 | | 40.66 | |
| phosphate, | | | | | | |
| anhydrous | | | | | | |
| Maize starch | 20.33 | | 20.00 | | 20.33 | |
| Sucrose | 5.0 | | 0.0 | | 0.0 | |
| Copovidone | 0.0 | | 5.0 | | 0.0 | |
| Maltodextrine | 0.0 | | 0.0 | | 3.35 | |
| Water, purified¹ | — | | — | | — | |
| Cellulose, | 25.2 | | 26.2 | | 26.0 | |
| microcrystalline | | | | | | |
| Croscarmellose | 3.0 | | 3.0 | | 3.0 | |
| sodium | | | | | | |
| Talc | 1.5 | | 1.5 | | 3.0 | |
| Magnesium | 1.0 | | 1.0 | | 1.0 | |
| stearate | | | | | | |
| Weight of tablet | 100 | mg | 100 | mg | 125 | mg |
| core | | | | | | |

The use of copovidone as binder (Formulation No. 6) leads to tablets with good pharmaceutical technical properties, e.g. a relative long disintegration time permitting the tablets to be swallowed as whole tablets (as demonstrated in table 10) and acceptable stability data (as demonstrated in table 11):

TABLE 10
Comparision of pharmaceutical technical data for tablets containing
compound of formula I succinate with the composition given in table 9.

| Pharm. | Weight of the | | Friability | Disintegration |
|---|---|---|---|---|
| Technical data | tablet core | Hardness | (16 min) | (sec.) |
| Form. 1 | 100 mg | 46 N | 0.5% | 11 |
| Form. 2 | 100 mg | 50 N | 0.6% | 22 |
| Form. 3 | 100 mg | 48 N | 0.5% | 35 |
| Form. 4 | 100 mg | 53 N | — | 39 |
| Form. 5 | 100 mg | 63 N | — | 45 |
| Form. 6 | 100 mg | 37 N | 0.5% | 112 |
| Form. 7 | 125 mg | 36 N | 0.7% | 43 |

Some differences in the stability of the products containing different binders can be seen in table 11 (next page).

TABLE 11
Decomposition of compound of formulation 1 to 6 -
different binders are used, composition of tablets
given in table 9

| | Total decomposition (%) of API | | | | | |
|---|---|---|---|---|---|---|
| Treatment | Form. 1 | Form. 2 | Form. 3 | Form. 4 | Form. 5 | Form. 6 |
| Initial analysis | ND | ND | ND | ND | ND | ND |
| Autoclavation | 0.43 | 0.44 | 0.94 | 0.51 | 0.99 | 0.53 |
| 80° C. for 48 | 2.6 | 3.2 | 9.7 | 3.4 | 1.4 | 5.4 |
| hours (open) | | | | | | |
| 80° C. for 48 | 5.3 | 1.7 | 5.2 | 2.0 | 1.9 | 5.9 |
| hours (closed) | | | | | | |
| 80° C. for 144 | 5.0 | 6.8 | 20.0 | 6.6 | 2.6 | 12.7 |
| hours (open) | | | | | | |
| 80° C. for 144 | 2.7 | 4.5 | 9.0 | 3.8 | 5.1 | 2.9 |
| hours (closed) | | | | | | |
| 40° C./ | 0.17 | 0.18 | 0.25 | 0.25 | 0.17 | 0.32 |
| 75% RH | | | | | | |
| for 1 week | | | | | | |
| 40° C./ | 0.18 | 0.28 | 0.34 | 0.30 | 0.25 | 0.31 |
| 75% RH | | | | | | |
| for 3 weeks | | | | | | |
| 40° C./ | 0.25 | 0.30 | 0.43 | 0.35 | 0.35 | 0.41 |
| 75% RH | | | | | | |
| for 6 weeks | | | | | | |
| 40° C./ | 0.30 | 0.36 | 0.70 | 0.38 | 0.54 | 0.66 |
| 75% RH | | | | | | |
| for 10 weeks | | | | | | |
| 40° C./ | 0.33 | 0.36 | 0.80 | 0.41 | 0.60 | 0.75 |
| 75% RH | | | | | | |
| for 12 weeks | | | | | | |
| 60° C. for 1 | 0.59 | 0.55 | 1.1 | 0.61 | 0.28 | 0.69 |
| week | | | | | | |
| 60° C. for 3 | 1.6 | 1.5 | 3.5 | 1.6 | 0.48 | 1.8 |
| weeks | | | | | | |
| 60° C. for 6 | 2.4 | 2.4 | 6.2 | 2.5 | 0.88 | 2.9 |
| weeks | | | | | | |
| 60° C. for 10 | 3.5 | 3.6 | 9.6 | 3.9 | 1.2 | 4.6 |
| weeks | | | | | | |
| 60° C. for 12 | 3.7 | 3.8 | 10.3 | 4.2 | 1.4 | 5.0 |
| weeks | | | | | | |

Decomposition of compound of formulation 7, in formulation
where maltodextrin is used as binder, composition of tablets
given in table 9

| Treatment | |
|---|---|
| Binder | Maltodextrin (form. 7) |
| Initial analysis | <0.05 |
| After autoclavation | 1.1 |
| 80° C. for 48 hours | 2.0 |
| 80° C. for 120 hours | 3.7 |
| 40° C./75% RH for 3 weeks | <0.05 |
| 60° C. for 3 weeks | 1.41 |

ND = Not detected

### EXAMPLE 3

### Preparation of Immediate Release Film-Coated Tablet Intended for Oral Administration III

Pharmaceutical Development

A study of the compatibility of the excipients and Compound I demonstrated that the components used in the tablet formulation were compatible with the compound. Based on this, a traditional wet granulation, tabletting and film-coating process was developed using standard methods and excipients.

Description of Drug Product

Compound I is formulated as immediate release film-coated tablet intended for oral administration. Tablets containing compound of formula I in this example are made in two strengths, 2.5 and 5 mg. The product containing compound of formula I is a white film-coated tablet encapsulated in a brownish red hard capsule. Other strengths, such as 2, 3, 4, 6, 7, 8, 9, 10, 11, 12, 13, or 14 mg, may be prepared in the same manner.

Composition

The compositions of the tablets 2.5 mg and 5 mg are given below in Table 12 and Table 13.

Manufacturing process and process controls is as in Example 1. A flow diagram of the manufacturing process and process controls is shown in FIG. 1.

TABLE 12
Composition of tablets 2.5 mg and 5 mg (calcium phosphate
formulation)

| | Quantity per Unit | | | | | Reference to |
|---|---|---|---|---|---|---|
| Name of Ingredient | 2.5 mg | | 5 mg | | Function | Standard¹ |
| DRUG SUBSTANCE | | | | | | |
| Compound I, succinate | 3.333 | mg | 6.667 | mg | Active ingredient | In-house spec. |
| Corresponding to Compound I | 2.5 | mg | 5 | mg | | |
| EXCIPIENTS | | | | | | |
| Tablet core: | | | | | | |
| Calcium hydrogen phosphate, | 40.000 | mg | 40.000 | mg | Filler | Ph. Eur. |
| anhydrous | | | | | | |
| Maize starch | 20.000 | mg | 20.000 | mg | Filler | Ph. Eur. |
| Copovidone | 5.00 | mg | 5.00 | mg | Binder | Ph. Eur. |
| Water, purified² | q.s. | | q.s. | | Granulation liquid | Ph. Eur. |
| Cellulose, microcrystalline | 26.17 | mg | 22.83 | mg | Filler | Ph. Eur. |
| Croscarmellose sodium | 3 | mg | 3 | mg | Disintegrant | Ph. Eur. |
| Talc | 1.5 | mg | 1.5 | mg | Lubricant | Ph. Eur. |
| Magnesium stearate | 1 | mg | 1 | mg | Lubricant | Ph. Eur. |
| Weight of each tablet core | 100 | mg | 100 | mg | | |
| Film-coating: | | | | | | |
| Opadry Y-1-7000 white | | | | | | |
| consisting of: | | | | | | |
| Hypromellose (5 mPa · s.) | 1.563 | mg | 1.563 | mg | Film former | Ph. Eur. |
| Macrogol 400 | 0.156 | mg | 0.156 | mg | Plasticizer | Ph. Eur. |
| Titanium dioxide (E171) | 0.781 | mg | 0.781 | mg | Pigment | Ph. Eur. |
| Water, purified² | q.s. | | q.s. | | Solvent | Ph. Eur. |
| Weight of each film-coated tablet | 102.5 | mg | 102.5 | mg | Lubricant | Ph. Eur. |

| | | |
|---|---|---|
| Magnesium stearate | q.s. | q.s. |

¹The current pharmacopoeia is used
²Volatile material

TABLE 13
Composition of tablets 2.5 mg and 5 mg (lactose formulation)

| | Quantity per Unit | | | | | Reference to |
|---|---|---|---|---|---|---|
| Name of Ingredient | 2.5 mg | | 5 mg | | Function | Standard¹ |
| DRUG SUBSTANCE | | | | | | |
| Compound I, succinate | 3.333 | mg | 6.667 | mg | Active ingredient | In-house spec. |
| Corresponding to Compound I | 2.5 | mg | 5 | mg | | |
| EXCIPIENTS | | | | | | |
| Tablet core: | | | | | | |
| Lactose | 39.330 | mg | 39.330 | mg | Filler | Ph. Eur. |
| Maize starch | 15.000 | mg | 15.000 | mg | Filler | Ph. Eur. |
| Copovidone | 3.35 | mg | 3.35 | mg | Binder | Ph. Eur. |
| Water, purified² | q.s. | | q.s. | | Granulation liquid | Ph. Eur. |
| Cellulose, microcrystalline | 34.99 | mg | 31.65 | mg | Filler | Ph. Eur. |
| Croscarmellose sodium | 3 | mg | 3 | mg | Disintegrant | Ph. Eur. |
| Magnesium stearate | 1 | mg | 1 | mg | Lubricant | Ph. Eur. |
| Weight of each tablet core | 100 | mg | 100 | mg | | |
| Film-coating: | | | | | | |
| Opadry Y-1-7000 white | | | | | | |
| consisting of: | | | | | | |
| Hypromellose (5 mPa · s.) | 1.563 | mg | 1.563 | mg | Film former | Ph. Eur. |
| Macrogol 400 | 0.156 | mg | 0.156 | mg | Plasticizer | Ph. Eur. |
| Titanium dioxide (E171) | 0.781 | mg | 0.781 | mg | Pigment | Ph. Eur. |

| | | | | |
|---|---|---|---|---|
| Water, purified² | q.s. | q.s. | Solvent | Ph. Eur. |

| | | | | |
|---|---|---|---|---|
| Weight of each film-coated tablet | 102.5 | mg | 102.5 | mg |

| | | | | |
|---|---|---|---|---|
| Magnesium stearate | q.s. | q.s. | Lubricant | Ph. Eur. |

¹The current pharmacopoeia is used
²Volatile material

1. A pharmaceutical composition comprising the compound of formula (I): 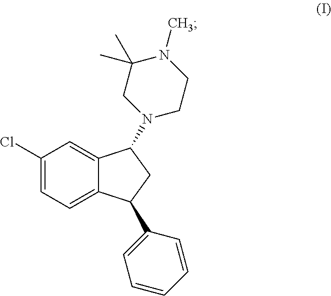
in a therapeutically effective amount of from 4-14 mg calculated as the free base.

2. The composition of claim 1, wherein the composition is formulated for oral administration.

3. The composition of claim 1, wherein the compound of formula (I) is in the form of a succinate or malonate salt.

4. The composition of claim 1, wherein the amount of the compound of formula (I) is 4-12 mg, 5-14 mg, 4-6 mg, 6-8 mg, 8-10 mg, 10-12 mg, 12-14 mg, 5-7 mg, 7-9 mg, 9-11 mg, 11-13 mg, 5 mg, 7 mg, 10 mg, or 14 mg.

5. The composition of claim 1, wherein the composition is for oral administration once daily.

6. The composition of claim 1, wherein the composition further comprises copovidone, as a binder.

7. The composition of claim 1, wherein the composition is for treatment of cognitive dysfunction, schizophrenia, Schizophreniform, Disorder, Schizoaffective Disorder, Delusional Disorder, Brief Psychotic Disorder, Shared Psychotic Disorder, mania in bipolar disorder, an anxiety disorder, depression maintenance of a bipolar disorder, a sleep disturbance, migraine, neuroleptic-induced parkinsonism, cocaine abuse, nicotine abuse, or alcohol abuse.

8. (canceled)

9. A method of treating cognitive dysfunction, schizophrenia, Schizophreniform Disorder, Schizoaffective Disorder, Delusional Disorder, Brief Psychotic Disorder, Shared Psychotic Disorder, mania in bipolar disorder, anxiety disorders, depression, maintenance of bipolar disorders, sleep disturbances, migraine, neuroleptic-induced parkinsonism, or cocaine abuse, nicotine abuse, or alcohol abuse, comprising administering a therapeutically effective amount of from 4-14 mg calculated as the free base of the compound of formula Ito a patient in need thereof.

10. A pharmaceutical composition comprising the compound of formula (I): 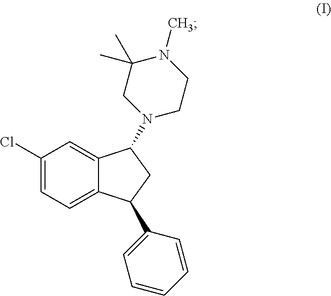
and povidone or copovidone as binder.

11. The composition of claim 10, wherein the binder is present in a concentration range of 2-10% (w/w).

12. The composition of claim 10, wherein the binder is Kollidone VA64.

13. The composition of claim 10, wherein the compound of formula (I) is in the form of a succinate salt.

14. The composition of claim 2, wherein the composition has the form of a tablet or capsule.

15. The composition of claim 11, wherein the concentration range is of 2-4% (w/w), 4-6% (w/w), 6-8% (w/w) or 8-10% (w/w).
